(19) United States
(12) Patent Application Publication
(10) Pub. No.: US 2026/0166249 A1
(43) Pub. Date: Jun. 18, 2026

(54) NASOPHARYNGEAL AIRWAY DEVICE

(71) Applicant: 6:8 Medical Solutions LLC, Myrtle Beach, SC (US)

(72) Inventor: Uriah Shane Popp, Havre De Grace, ME (US)

(73) Assignee: 6:8 Medical Solutions LLC, Myrtle Beach, SC (US)

(21) Appl. No.: 19/417,820

(22) Filed: Dec. 12, 2025

Related U.S. Application Data
(60) Provisional application No. 63/730,989, filed on Dec. 12, 2024.

(51) Int. Cl. A61M 16/04; A61M 25/02

(52) U.S. Cl. CPC A61M 16/0461; A61M 2025/024; A61M 2210/0618; A61M 2210/065

(57) ABSTRACT

A nasopharyngeal airway device includes a tubular body defining a hollow lumen extending from a proximal end to a distal end. The tubular body is configured for insertion through a nostril of a patient to establish an airway passage through the nasopharynx. The device further includes a distal opening formed at the distal end of the tubular body. The distal opening has a beveled shape. The device further includes one or more fenestrations formed in a wall of the tubular body at a location proximal to the distal opening. The fenestrations provide an auxiliary airflow passage through the wall of the tubular body. The device also includes a securing clip coupled to the proximal end of the tubular body. The securing clip is configured to engage external nasal anatomy of the patient to resist axial rotation of the tubular body and inhibit withdrawal of the tubular body from the nostril.

### CROSS-REFERENCE TO RELATED APPLICATIONS

This Application claims priority to U.S. Provisional Ser. No. 63/730,989 filed on Dec. 12, 2024, entitled “SurSecure NPA (Nosopharyngeal Airway-NPA,” the contents of which are incorporated herein by reference.

### BACKGROUND

### Field of the Art

The present invention is related to the field of medical airway management devices, and more particularly to a nasopharyngeal airway having improved securing mechanisms and enhanced patency features for maintaining unobstructed respiratory function in patients requiring airway intervention.

### Discussion of the State of the Art

Nasopharyngeal airways are medical devices used to establish and maintain a patent airway in patients who are unconscious, semi-conscious, or otherwise unable to maintain adequate respiratory function. These devices are inserted through the nasal passage and extend into the pharynx, bypassing potential obstructions caused by the tongue or soft palate. Nasopharyngeal airways are particularly valuable in emergency medicine, trauma care, and surgical settings where rapid airway management is critical to patient survival.

Conventional nasopharyngeal airway devices typically comprise a curved tube with a trumpet-shaped or flared proximal end and a beveled distal tip. Despite their widespread use and clinical importance, conventional nasopharyngeal airways suffer from numerous deficiencies that compromise their effectiveness and reliability. These shortcomings present significant challenges for healthcare providers and can result in adverse patient outcomes.

One persistent problem with existing nasopharyngeal airways is unwanted rotation during insertion or subsequent patient movement. When the device rotates, the beveled distal end can shift away from its intended midline position toward the soft tissue of the oral pharynx. This rotation can cause the distal bevel to press against mucosal tissue, resulting in partial or complete occlusion of airflow through the device. In emergency situations where every breath is critical, such occlusion can have severe consequences for patient oxygenation and survival.

Another significant deficiency relates to device migration and inadequate securement. Conventional nasopharyngeal airways lack any inherent mechanism to prevent the device from backing out of the nasal passage during patient transport, routine handling, or involuntary patient movements. Healthcare providers are forced to rely on medical tape applied to the patient's face to secure the device in position. However, tape adhesion to facial skin is notoriously unreliable, particularly in trauma scenarios where blood, perspiration, mucus, or other bodily fluids are present on the skin surface. The failure of tape to maintain adequate adhesion can result in device displacement at critical moments, potentially compromising the patient's airway when immediate intervention may not be possible.

Conventional nasopharyngeal airways also lack any visual indicator for bevel orientation. Once the device has been inserted beyond the base of the tongue, the distal end is no longer visible to the clinician. The traditional trumpet-shaped proximal end is circular and symmetrical, providing no external reference point that corresponds to the orientation of the distal bevel. Consequently, healthcare providers have no reliable means of verifying whether the distal bevel remains properly oriented after insertion. This uncertainty can lead to undetected malposition and compromised airway patency.

The design of existing nasopharyngeal airways provides no redundancy for maintaining airflow in the event of distal obstruction. The single beveled opening at the distal end represents the sole pathway for air exchange. When this opening becomes blocked by blood, mucus, vomitus, or compression against soft tissue, airflow through the device is completely eliminated. The device then fails entirely in its fundamental purpose of maintaining a patent airway, potentially without any indication to the clinician that obstruction has occurred.

Furthermore, the traditional trumpet-shaped proximal end of conventional nasopharyngeal airways can interfere with proper insertion depth. The tapered, flared design of the trumpet creates a geometry that naturally tends to force the device outward rather than allowing it to seat at the appropriate anatomical depth. This design limitation can result in suboptimal positioning of the distal tip relative to the pharyngeal airway, reducing the effectiveness of the device in maintaining airway patency.

The cumulative effect of these deficiencies is that conventional nasopharyngeal airways require additional materials and interventions to function adequately, provide no feedback to clinicians regarding proper placement and orientation, offer no protection against rotation or migration, and lack any backup mechanism for maintaining airflow when the primary distal opening becomes obstructed. These limitations represent significant obstacles to reliable airway management in clinical practice.

### SUMMARY

A nasopharyngeal airway device (also called a nasopharyngeal airway) in accordance with the present invention includes a tubular curved body having a distal end and a proximal end. The distal end has a bevel-shaped distal opening and the proximal end includes a securing clip attached thereto. The tubular body further includes openings or fenestrations that pass through the sidewall thereof.

The nasopharyngeal airway in accordance with the present invention addresses limitations of conventional nasopharyngeal airways through an integrated design incorporating a nasal securing mechanism, visual orientation indication, and redundant airflow pathways. The nasopharyngeal airway in accordance with the present invention delivers substantial benefits across multiple dimensions of clinical care, patient safety, and operational efficiency.

A primary benefit of the nasopharyngeal airway is significantly improved airway security and reliability, particularly in high-stress emergency, prehospital, and military environments where continuous monitoring of airway devices may not be feasible. The nasal securing clip positively engages the external nasal anatomy to prevent both axial rotation and backward migration of the airway tube, eliminating common failure modes associated with conventional nasopharyngeal airways. By maintaining the device in its proper position and orientation, the nasopharyngeal airway in accordance with the present invention reduces the risk of airway compromise that could result from device displacement or rotation during patient transport, movement, or resuscitation efforts.

The fenestrations positioned proximal to the distal tip provide backup airflow passages that maintain airway patency even when the primary distal opening becomes occluded by blood, mucus, or soft tissue. This redundant airflow passageway provided by the fenestrations represents a significant safety advancement, as occlusion of conventional nasopharyngeal airways can lead to complete airway obstruction without immediate visual indication to the clinician.

The self-securing design of the nasopharyngeal airway eliminates the need for adhesive tape or other supplemental securing methods traditionally required to maintain nasopharyngeal airway position. This benefit reduces the time required for airway management procedures and decreases dependence on additional supplies that may be limited in emergency or austere environments. The securing clip functions as an integrated depth stop, ensuring proper insertion depth without requiring clinician estimation or additional measurement steps.

The securing clip provides immediate visual confirmation of proper bevel orientation within the nasal passage, allowing clinicians to verify correct placement simply by observing the clip's position on the external nose. This visual indication capability eliminates the need for additional assessment procedures to confirm bevel positioning and reduces the cognitive burden on clinicians during high-stress resuscitation scenarios. The orientation indication functions properly regardless of which nostril receives the device, providing consistent visual feedback in all clinical applications.

The securing clip may be manufactured as a standalone accessory compatible with existing standard nasopharyngeal airways, allowing healthcare providers and emergency medical systems to retrofit their current inventory without complete device replacement. This compatibility benefit reduces implementation costs and allows immediate adoption of the securing and orientation indication benefits across existing equipment stocks.

The nasopharyngeal airway may be made from a semi-flexible material such that the device maintains its manufactured shape, but is able to bend upon application of force. For example, the nasopharyngeal airway may be made of silicone, which provides biocompatibility and flexibility that enhances patient comfort while reducing trauma during insertion. The proximal end of the nasopharyngeal airway is flat rather than having a flared configuration. The flat proximal end allows complete seating of the device within the nostril at proper depth, eliminating the external protrusion associated with conventional trumpet-shaped proximal ends. Optional lubricious or hydrophilic surface coatings may further reduce insertion resistance and associated tissue trauma.

The securing clip incorporates a relieved section that accommodates variation in nostril size and nasal septum thickness across patient populations, allowing effective securing across a range of patient anatomies without requiring multiple device configurations or sizes for the securing mechanism.

In one example, the present invention is a nasopharyngeal airway device that includes a tubular body defining a hollow lumen extending from a proximal end to a distal end. The tubular body is configured for insertion through a nostril of a patient to establish an airway passage through the nasopharynx. The tubular body may have an outer diameter that is uniform along an entire length of the tubular body. The tubular body may be semi-flexible and may have a curved shape having a convex side and a concave side. The device further includes a distal opening formed at the distal end of the tubular body. The distal opening has a beveled shape. The device further includes one or more fenestrations formed in a wall of the tubular body at a location proximal to the distal opening. The fenestrations provide an auxiliary airflow passage through the wall of the tubular body. There may be a plurality of fenestrations in the wall of the tubular body. The fenestrations may be positioned on the convex side of the tubular body. Still further, the device includes a securing clip coupled to the proximal end of the tubular body. The securing clip is configured to engage external nasal anatomy of the patient to resist axial rotation of the tubular body and inhibit withdrawal of the tubular body from the nostril. The securing clip may be integrally molded with the tubular body as a unitary structure, or may be a separate component configured to be removably attached to the tubular body. The securing clip may have a position that creates a space between the securing clip and the tubular body. The external nasal anatomy of the patient may be positioned in the space when the distal end of the tubular body is positioned in the nasopharynx. The securing clip may have a body portion that extends radially outward from the tubular body, and an engagement member that extends perpendicularly to the body portion. The external nasal anatomy of the patient may be frictionally engaged between the engagement member and the tubular body when the distal end of the tubular body is positioned in the nasopharynx.

In another example, the present invention is a nasopharyngeal airway device that includes a tubular body having a proximal end with a flat terminal surface and a distal end with a beveled opening. The tubular body defines a hollow lumen therethrough for airflow. The tubular body may have an outer diameter that is uniform along an entire length of the tubular body. The tubular body may be semi-flexible and may have a curved shape that includes a convex side and a concave side. The device further includes a plurality of fenestrations formed through a wall of the tubular body at positions proximal to the beveled opening. The plurality of fenestrations are configured to permit airflow when the beveled opening is obstructed. The plurality of fenestrations may be positioned on the convex side of the tubular body. The device further includes a securing clip coupled to the proximal end of the tubular body. The securing clip has a body portion that extends radially outward from the tubular body, and an engagement member that extends perpendicularly to the body portion. External nasal tissue of a patient is frictionally engaged between the engagement member and the tubular body when the beveled opening is in the patient's nasopharynx. The securing clip may be integrally molded with the tubular body as a unitary structure, or may be a separate component configured to be removably attached to the tubular body.

### BRIEF DESCRIPTION OF THE DRAWINGS

The accompanying drawings illustrate several embodiments and, together with the description, serve to explain the principles of the invention according to the embodiments. It will be appreciated by one skilled in the art that the particular arrangements illustrated in the drawings are merely exemplary and are not to be considered as limiting of the scope of the invention or the claims herein in any way.

FIG. 1 is a plan view of a nasopharyngeal airway, in accordance with an embodiment of the present invention.

FIG. 2 is a close up view of a proximal portion of a nasopharyngeal airway, in accordance with an embodiment of the present invention.

FIG. 3 is a close up view of a distal portion of a nasopharyngeal airway, in accordance with an embodiment of the present invention.

FIG. 4 depicts a nasopharyngeal airway in use positioned in a nasal cavity of a patient, in accordance with an embodiment of the present invention.

FIGS. 5 and 6 are plan views of a nasopharyngeal airway and a securing clip in a disassembled state and an assembled state, respectively, in accordance with an embodiment of the present invention.

FIG. 7 depicts an assembly comprising a nasopharyngeal airway and a securing clip in use positioned in a nasal cavity of a patient, in accordance with an embodiment of the present invention.

### DETAILED DESCRIPTION

The present invention is a nasopharyngeal airway that includes a tubular body having a proximal end and a distal end. The distal end includes a distal opening having a beveled shape and the proximal end includes a securing clip attached thereto. The nasopharyngeal airway further includes fenestrations in the sidewall of the tubular body. The fenestrations provide an additional airway passage for redundancy in case the distal opening becomes occluded.

The invention is described by reference to various elements herein. It should be noted, however, that although the various elements of the inventive apparatus are described separately below, the elements need not necessarily be separate. The various embodiments may be interconnected and may be cut out of a singular block or mold. The variety of different ways of forming an inventive apparatus, in accordance with the disclosure herein, may be varied without departing from the scope of the invention.

One or more different embodiments may be described in the present application. Further, for one or more of the embodiments described herein, numerous alternative arrangements may be described; it should be appreciated that these are presented for illustrative purposes only and are not limiting of the embodiments contained herein or the claims presented herein in any way. One or more of the arrangements may be widely applicable to numerous embodiments, as may be readily apparent from the disclosure. In general, arrangements are described in sufficient detail to enable those skilled in the art to practice one or more of the embodiments, and it should be appreciated that other arrangements may be utilized and that structural, logical, software, electrical and other changes may be made without departing from the scope of the embodiments. Particular features of one or more of the embodiments described herein may be described with reference to one or more particular embodiments or figures that form a part of the present disclosure, and in which are shown, by way of illustration, specific arrangements of one or more of the aspects. It should be appreciated, however, that such features are not limited to usage in the one or more particular embodiments or figures with reference to which they are described. The present disclosure is neither a literal description of all arrangements of one or more of the embodiments nor a listing of features of one or more of the embodiments that must be present in all arrangements.

Headings of sections provided in this patent application and the title of this patent application are for convenience only and are not to be taken as limiting the disclosure in any way.

Devices that are in communication with each other need not be in continuous communication with each other, unless expressly specified otherwise. In addition, devices that are in communication with each other may communicate directly or indirectly through one or more communication means or intermediaries, logical or physical.

A description of an aspect with several components in communication with each other does not imply that all such components are required. To the contrary, a variety of optional components may be described to illustrate a wide variety of possible embodiments and in order to more fully illustrate one or more embodiments. Similarly, although process steps, method steps, algorithms or the like may be described in a sequential order, such processes, methods and algorithms may generally be configured to work in alternate orders, unless specifically stated to the contrary. In other words, any sequence or order of steps that may be described in this patent application does not, in and of itself, indicate a requirement that the steps be performed in that order. The steps of described processes may be performed in any order practical. Further, some steps may be performed simultaneously despite being described or implied as occurring non-simultaneously (e.g., because one step is described after the other step). Moreover, the illustration of a process by its depiction in a drawing does not imply that the illustrated process is exclusive of other variations and modifications thereto, does not imply that the illustrated process or any of its steps are necessary to one or more of the embodiments, and does not imply that the illustrated process is preferred. Also, steps are generally described once per aspect, but this does not mean they must occur once, or that they may only occur once each time a process, method, or algorithm is carried out or executed. Some steps may be omitted in some embodiments or some occurrences, or some steps may be executed more than once in a given aspect or occurrence.

When a single device or article is described herein, it will be readily apparent that more than one device or article may be used in place of a single device or article. Similarly, where more than one device or article is described herein, it will be readily apparent that a single device or article may be used in place of the more than one device or article.

The functionality or the features of a device may be alternatively embodied by one or more other devices that are not explicitly described as having such functionality or features. Thus, other embodiments need not include the device itself.

Techniques and mechanisms described or referenced herein will sometimes be described in singular form for clarity. However, it should be appreciated that particular embodiments may include multiple iterations of a technique or multiple instantiations of a mechanism unless noted otherwise. Process descriptions or blocks in figures should be understood as representing modules, segments, or portions of code which include one or more executable instructions for implementing specific logical functions or steps in the process. Alternate implementations are included within the scope of various embodiments in which, for example, functions may be executed out of order from that shown or discussed, including substantially concurrently or in reverse order, depending on the functionality involved, as would be understood by those having ordinary skill in the art.

The detailed description set forth herein in connection with the appended drawings is intended as a description of various configurations and is not intended to represent the only configurations in which the concepts described herein may be practiced. The detailed description includes specific details for the purpose of providing a thorough understanding of various concepts. However, it will be apparent to those skilled in the art that these concepts may be practiced without these specific details. In some instances, well known structures and components are shown in block diagram form in order to avoid obscuring such concepts.

### Apparatus

The present invention is for a nasopharyngeal airway device comprising several integrated components designed to maintain airway patency and provide secure positioning within the nasal passage. In particular, the nasopharyngeal airway includes a tubular body serving as the main structural element that provides a hollow passage for airflow from the nostril through the nasopharynx to beyond the base of the tongue. When the nasopharyngeal airway is in the correct position, the tubular body extends from a proximal end that remains external to the nostril to a distal end that terminates in the oral pharynx. An anti-rotational nasal securing clip is attached to the proximal end of the tubular body. The securing clip engages the external nasal anatomy to prevent axial rotation, inhibit withdrawal or migration, ensure proper insertion depth, stabilize the device during patient movement, and simultaneously serve as a visual indicator for distal bevel orientation. A distal beveled opening comprising an angled cut at the distal end of the tubular body serves as the primary airway aperture positioned beyond the base of the tongue in the oral pharynx. The bevel orientation of the distal end is directed toward the midline of the trachea to maintain airway patency. Fenestrations comprising one or more openings positioned in the wall of the tubular body proximal to the distal beveled opening provide backup airflow passages in the event that the primary distal aperture becomes obstructed by blood, mucus, or soft tissue. The proximal end of the tubular body is flat rather than trumpet-shaped, allowing the device to seat completely within the nostril at proper depth. A lubricious coating or hydrophilic treatment may be applied to the exterior surface of the tubular body to facilitate smooth insertion and reduce tissue trauma.

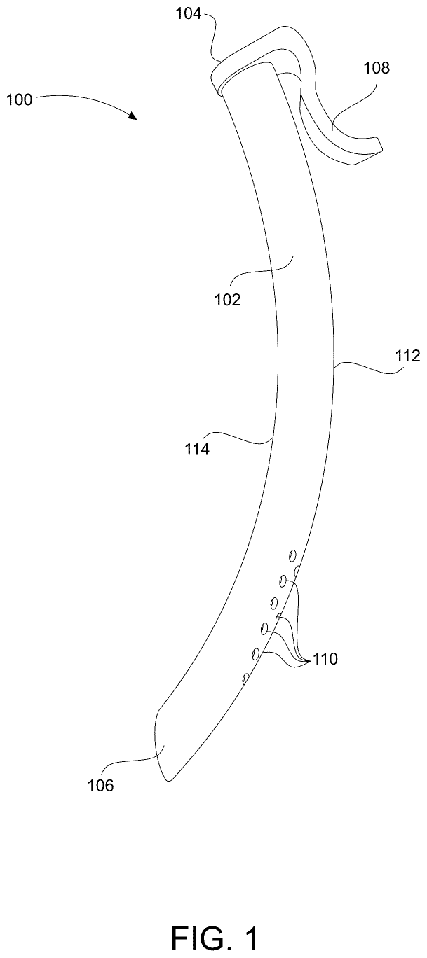

A nasopharyngeal airway 100 in accordance with the present invention is depicted in FIG. 1. The device 100 includes a tubular body 102 having a proximal end 104 and a distal end 106. Attached to the proximal end 104 is a nasal securing clip 108 configured for securing the device to the patient's nostril once the device is inserted through the patient's nasal passage. The tubular body 102 further includes a plurality of fenestrations 110, or openings, that extend through the sidewall of the tubular body 102.

The tubular body 102 forms the primary structural element of the nasopharyngeal airway device 100. The tubular body 102 comprises an elongated hollow cylindrical member having a continuous lumen extending from the proximal end 104 to the distal end 106. The tubular body 102 is fabricated from a medical-grade semi-flexible polymeric material such that the tubular body 102 retains its manufactured shape but is able to bend upon application of force. For example, the tubular body 102 may be made of silicone, polyvinyl chloride, latex, thermoplastic elastomer, or the like, selected to provide sufficient flexibility for comfortable insertion and conformance to nasal and pharyngeal anatomy while maintaining adequate structural integrity to prevent lumen collapse during use. Further in conformance to nasal and pharyngeal anatomy, the tubular body 102 has a curved shape that includes a convex side 112 and a concave side 114. The exterior surface of the tubular body 102 may optionally incorporate a lubricious coating or hydrophilic surface treatment to reduce frictional resistance during insertion.

The tubular body 102 has an outer diameter sized to correspond with standard nasopharyngeal airway sizing conventions, typically ranging from approximately 6 millimeters to approximately 9 millimeters for adult applications, with proportionally smaller sizes available for pediatric applications. The inner diameter defines the airflow lumen and is sized to provide adequate airflow capacity while accommodating wall thickness sufficient for structural integrity. The length of the tubular body 102 is selected based on patient anatomy, typically ranging from approximately 10 centimeters to approximately 18 centimeters, to ensure the distal end 106 reaches the oral pharynx beyond the base of the tongue when the device 100 is properly inserted.

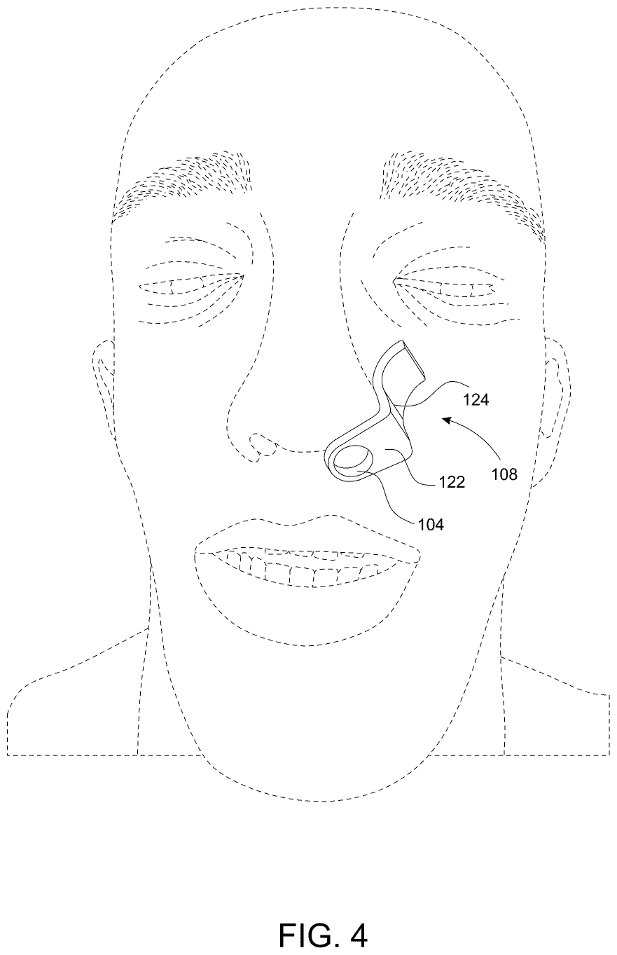

The proximal end 104 of the tubular body 102 comprises a flat terminal configuration rather than the trumpet-shaped or flared configuration found in conventional nasopharyngeal airways. In other words, the outer diameter of the tubular body 102 is fixed for the entire length of the tubular body 102 and does not expand or flare at the proximal end 104. This flat proximal end 104 comprises a substantially planar surface oriented perpendicular to the longitudinal axis of the proximal portion of the tubular body 102. The flat configuration allows the proximal end 104 to seat completely within the nostril opening or flush with the external naris when the device 100 is inserted to the proper depth, as shown in FIG. 4. The flat proximal end 104 eliminates the external protrusion associated with trumpet-shaped designs, reducing the risk of accidental dislodgement during patient handling, transport, or positioning changes.

The flat design of the proximal end 104 operates synergistically with the securing clip 108 to maintain proper device positioning throughout use. By eliminating the outward expulsion forces caused by the trumpet-shaped proximal end of the conventional device, the flat profile of the proximal end 104 reduces the retention burden on the securing clip 108, while the securing clip 108 provides supplemental retention that accommodates patient movement and other displacement forces.

The proximal end 104 includes a proximal opening that communicates with the internal lumen of the tubular body 102, providing the external airflow aperture through which ambient air enters the device 100 during patient inspiration. The proximal opening may be circular, oval, or otherwise shaped to maximize airflow area while conforming to the flat terminal configuration.

The distal end 106 of the tubular body 102 terminates in a beveled opening that serves as the primary airway aperture. The distal beveled opening is defined by a planar cut through the tubular wall at an oblique angle relative to the longitudinal axis of the distal portion of the tubular body 102, thereby creating an elliptical or ovoid aperture geometry when viewed in plan. This beveled configuration creates a larger effective opening area compared to a perpendicular cut, facilitating airflow and reducing the likelihood of complete obstruction. The bevel angle of the distal beveled opening is angled between approximately 30 degrees and approximately 60 degrees relative to the longitudinal axis of the distal portion of the tubular body 102. Selection of the bevel angle within this range balances competing design considerations including aperture area maximization, insertion ease, and manufacturing reproducibility.

Alternatively, curved bevel configurations may be employed wherein the cut surface follows an arcuate rather than planar path. The peripheral edge of the distal beveled opening may incorporate rounded or radiused edges to minimize sharp contact surfaces against tissue. Reinforced rim structures may be provided around the perimeter of the distal beveled opening to maintain aperture geometry and resist deformation. Flexible lip portions extending from the edge of the distal beveled opening may be configured to deflect upon tissue contact while maintaining aperture patency.

The bevel orientation is configured such that when the device 100 is properly inserted, the beveled opening faces toward the midline of the patient's trachea, directing inspired air toward the laryngeal inlet and trachea. The distal tip formed by the beveled cut is rounded and atraumatic to minimize tissue injury during insertion and positioning.

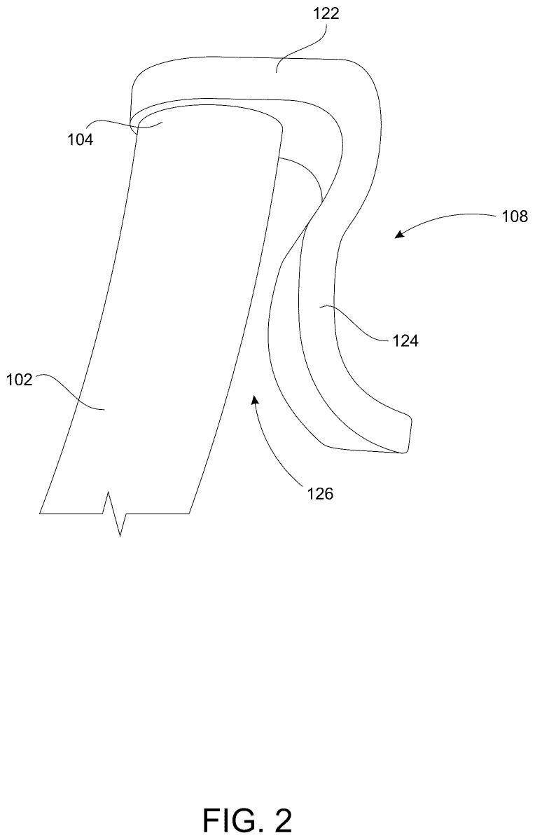

The anti-rotational nasal securing clip 108 is designed to engage the external nasal anatomy, specifically the alar cartilages or the outer surface of the nostril, to provide multi-functional stabilization of the device 100. The securing clip 108, shown in more detail in FIG. 2, comprises a body portion 122 that attaches to or is integral with the tubular body 102 and that projects radially outward from the tubular body 102. The securing clip 108 further includes an engagement member 124 that extends substantially perpendicularly from the body portion 122 to contact and engage external nasal structures. In one embodiment, the engagement member 124 comprises a substantially U-shaped or C-shaped configuration that creates a narrow space 126 between the engagement member 124 and the tubular body 102. The curvature of the engagement member 124 substantially conforms to the external nasal geometry, thereby distributing contact pressure across a broader surface area and enhancing patient comfort during extended use. The engagement member 124 may incorporate a relieved section or cutout region that accommodates anatomical variations in nostril size, shape, and tissue thickness across different patient populations. As shown in FIG. 4, the outer surface of the nostril is positioned in the space 126 when the device 100 is in use such that there is a friction fit engagement between the securing clip 108 and the patient's nostril.

The securing clip 108 thereby functions as a depth stop that prevents both over-insertion beyond the intended pharyngeal positioning and under-insertion that would result in inadequate airway patency. The geometric relationship between the securing clip 108 orientation and the distal bevel is fixed and predetermined. This fixed angular relationship enables clinicians to visually confirm proper bevel positioning by observing the external orientation of the securing clip 108 without requiring direct visualization of the distal tip 106.

The securing clip 108 may be integrally molded with the tubular body 102 as a unitary structure, formed from the same polymeric material during the manufacturing process. Alternatively, the securing clip 108 may be fabricated as a separate component that attaches to the tubular body 102 through mechanical engagement such as snap-fit connection, friction fit, adhesive bonding, or other attachment mechanisms. In embodiments where the securing clip 108 is a separate component, it may be provided as an accessory that can be retrofitted to existing standard nasopharyngeal airway devices lacking integral securing mechanisms.

The securing clip 108 functions as a visual orientation indicator that provides external visual confirmation of the internal bevel orientation. Additionally or alternatively, the device 100 may include a visual bevel orientation indicator such as a marking, color coding, arrow, or geometric feature on the proximal end 104 of the device 100 that corresponds to the direction of the beveled opening at the distal end 106. When the visual indicator is properly aligned with external anatomical landmarks, the clinician can confirm that the distal bevel is oriented toward the midline of the trachea without requiring visualization of the distal tip 106.

The anti-rotational function of the securing clip 108 is achieved through the engagement of the engagement member 124 with the external nasal anatomy, as shown in FIG. 4. The geometry of the securing clip 108 prevents rotation of the tubular body 102 about its longitudinal axis because such rotation would require the engagement member 124 to move against or through the engaged nasal structures. The securing clip geometry may include non-circular profiles, asymmetric configurations, or textured surfaces that enhance rotational resistance.

The anti-migration function is achieved through the same engagement mechanism. Once the securing clip 108 is seated against the external nasal anatomy, withdrawal of the device 100 requires overcoming the resistance created by the engagement member 124. The engagement member 124 may include rearwardly-directed barbs, ridges, or friction-enhancing surfaces that permit advancement during insertion but resist withdrawal.

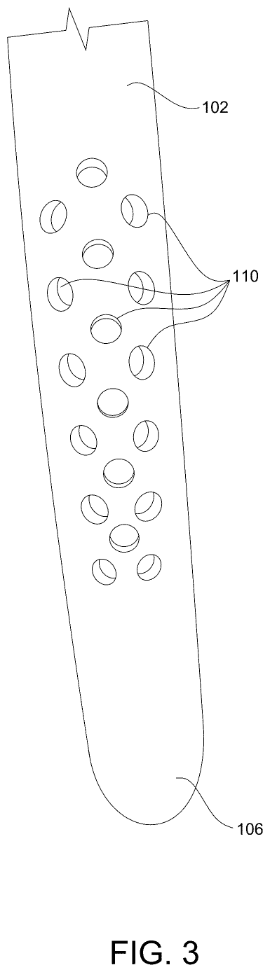

The fenestrations 110, shown in more detail in FIG. 3, comprise one or more openings formed through the wall of the tubular body 102 at locations proximal to the distal end 106. The fenestrations 110 may be positioned in the convex side 112 of the tubular body 102 These fenestrations 110 provide backup airflow passages that maintain airway patency when the primary distal aperture becomes obstructed by blood, mucus, soft tissue, or other materials.

The fenestrations 110 are positioned along the tubular body 102 at a distance of approximately 1 centimeter to approximately 3 centimeters proximal to the distal end 106. This positioning ensures that the fenestrations 110 are located within the pharyngeal airway space when the device 100 is properly inserted, thereby allowing the fenestrations 110 to provide effective backup airflow. The rear fenestrations 110 may be disposed on the tubular body wall at a circumferential position opposite to the bevel direction of the beveled distal end 106. This geometric relationship provides functional redundancy because tissue contact sufficient to occlude the bevel typically occurs on the bevel-facing side of the tubular body 102, leaving the opposite side where the rear fenestrations 110 are located exposed to the open airway cavity.

The fenestrations 110 are dimensioned such that their aggregate cross-sectional area provides airflow capacity equivalent to or approaching that achievable through the primary distal opening. This dimensional relationship ensures that occlusion of the distal opening does not substantially diminish the overall gas exchange capability of the device 100. The edges defining each of the fenestrations 110 may be rounded, chamfered, deburred, or otherwise smoothed to minimize tissue irritation and reduce the potential for mucosal trauma during insertion, positioning, or extended placement.

The individual apertures comprising the fenestrations 110 may assume various geometric configurations including circular, oval, elliptical, slotted, rectangular, or irregular shapes depending on manufacturing considerations and desired flow characteristics. The shape, layout, and dimensions of the fenestrations 110 are selected to provide adequate airflow capacity while maintaining structural integrity of the tubular body wall. In one embodiment, the fenestrations 110 comprise two or more circular openings having diameters of approximately 2 millimeters to approximately 4 millimeters, arranged in a distal portion of the convex side 112 of the tubular body 102. In another embodiment, the fenestrations comprise one or more elongated slots oriented parallel to the longitudinal axis of the tubular body 102. The quantity, dimensional parameters, spacing intervals, and distribution pattern of the fenestrations 110 may be varied to accommodate specific clinical applications, patient populations, or airway management protocols.

The edges of the fenestrations 110 may be rounded and smooth to prevent tissue injury and to reduce the accumulation of secretions at the fenestration margins. The fenestrations 110 may be formed during the initial molding of the tubular body 102 or may be created through secondary operations such as drilling, punching, or laser cutting.

The interconnection of the various components is achieved through the unitary construction of the tubular body 102 with the flat proximal end 104 and distal beveled opening in the distal end 106 formed as integral features. The rear fenestrations 110 are formed through the wall of the tubular body 102 at the predetermined locations. The securing clip 108, whether integrally molded or separately attached, is connected to the tubular body 102 at the predetermined position near the proximal end 104.

In embodiments with an integral securing clip 108, such as those depicted in FIGS. 1-4, the clip 108 is formed as a continuous extension of the tubular body material during the molding process. The transition between the tubular body 102 and the securing clip 108 is smooth and continuous, without joints or seams that could harbor bacteria or cause tissue irritation. When integrally molded, the proximal end 104 and securing clip 108 form a monolithic structure that reduces component count and assembly requirements.

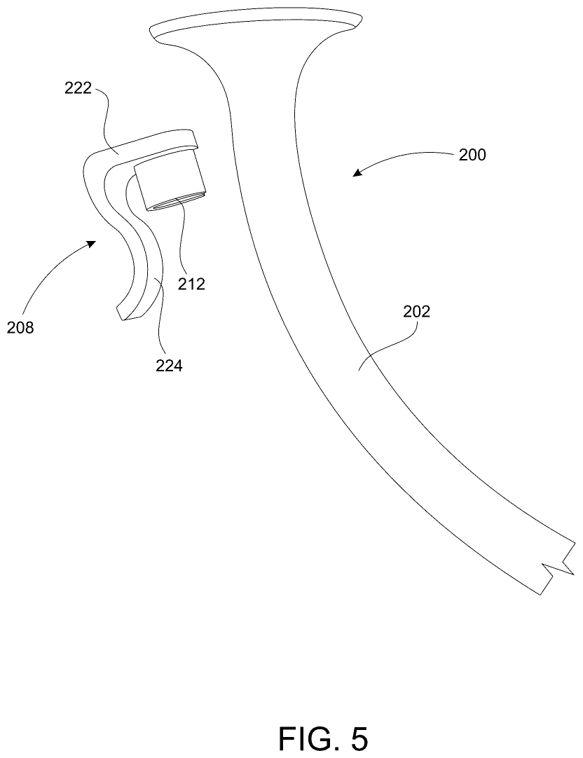

As shown in FIGS. 5-7, the securing clip 208 may be a separate piece that includes a central aperture or channel 212 sized to receive the tubular body 202. The clip 208 may be slid onto the tubular body 202 and positioned at the desired location, where it is retained by friction fit, snap-fit engagement with a corresponding feature on the tubular body 202, or adhesive bonding. The separate clip 208 allows for adjustment of the insertion depth by repositioning the clip 208 along the tubular body 202 prior to use. The separate clip 208 can be used with a conventional nasopharyngeal airway device 200 in order to ensure the device 200 is properly positioned and to hold the device 200 in place. When manufactured as a standalone component, the securing clip 208 provides equivalent securing, anti-rotation, and bevel indication functions to conventional trumpet-style nasopharyngeal airways.

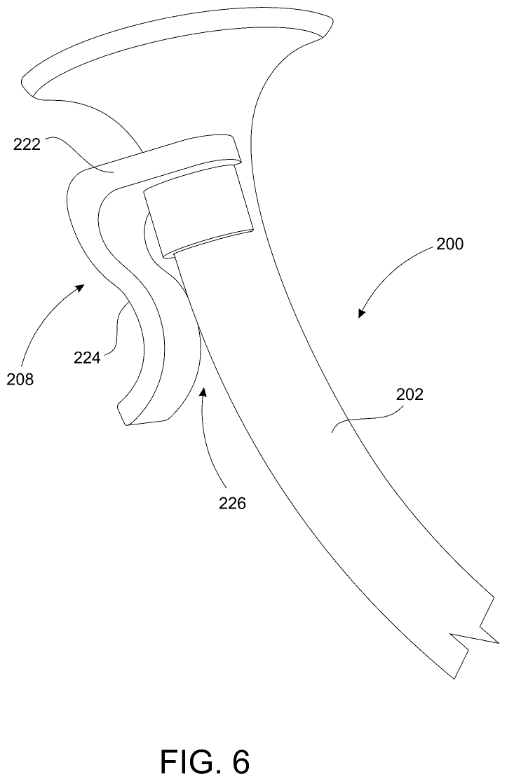

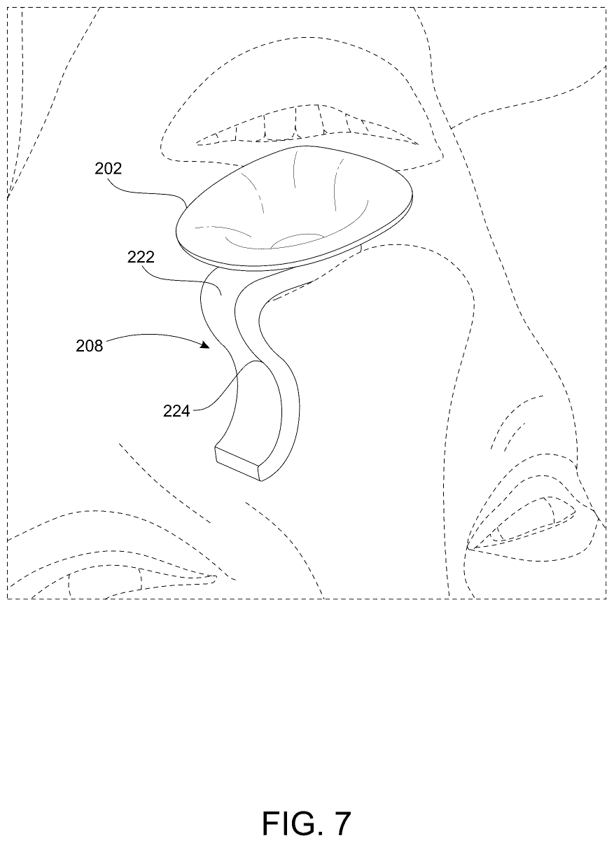

The separate securing clip 208 includes a main body portion 222 that extends radially outward from the aperture 212. The separate securing clip 208 further includes an engagement member 224 that extends substantially perpendicularly from the body portion 222 to contact and engage external nasal structures. The engagement member 224 may have a substantially U-shaped or C-shaped configuration that creates a narrow space 226 (see FIG. 6) between the engagement member 224 and the tubular body 202 to which the securing clip 208 is attached. The curvature of the engagement member 224 substantially conforms to the external nasal geometry, thereby distributing contact pressure across a broader surface area and enhancing patient comfort during extended use. The engagement member 224 may incorporate a relieved section or cutout region that accommodates anatomical variations in nostril size, shape, and tissue thickness across different patient populations. As shown in FIG. 7, the outer surface of the nostril is positioned in the space 226 when the device 200 is in use such that there is a friction fit engagement between the securing clip 208 and the patient's nostril.

The material selection for the various components considers biocompatibility, flexibility, durability, and manufacturing considerations. The tubular body 102 may be fabricated from semi-flexible medical-grade polymers that provide adequate flexibility for insertion while maintaining lumen patency. That is, the tubular body 102 may be made of a material that allows the device 100 to maintain its manufactured shape, but that is able to bend upon application of force. The securing clip 108, 208 may be fabricated from the same material as the tubular body 102 or from a different material having greater rigidity to enhance the stabilization function.

In use, the nasopharyngeal airway device 100 is inserted through the patient's nostril with the beveled opening oriented toward the nasal septum during initial insertion. The device 100 is advanced until the external nasal anatomy is positioned in the space 126 between the securing clip 108 and the tubular body 102, at which point further advancement is prevented and the proper insertion depth is achieved. The securing clip 108 acts as a visual orientation indicator by being aligned with external anatomical landmarks to confirm proper bevel orientation. Once inserted, the securing clip 108 prevents rotation and migration of the device 100, while the fenestrations 110 provide backup airflow if the primary distal aperture becomes obstructed.

The combination of the flat proximal end 104, anti-rotational securing clip 108, and fenestrations 110 provides a nasopharyngeal airway device 100 with enhanced stability, improved depth control, visual confirmation of bevel orientation, and backup airflow capability not found in conventional devices.

The nasopharyngeal airway device 100 interacts with several anatomical structures during placement and operation. Upon insertion, the curved, semi-flexible tubular body 102 traverses the nasal passage, following the natural curvature of the nasal cavity. The beveled distal tip is oriented toward the nasal septum to reduce contact with the more vascular lateral nasal wall and inferior turbinate during advancement. The tubular body 102 continues through the posterior nasal cavity until the distal end 106 is positioned in the nasopharynx, beyond the base of the tongue. This positioning establishes an unobstructed air channel that bypasses potential soft tissue obstruction caused by the tongue or pharyngeal tissues.

The securing clip 108 interfaces with the external nasal anatomy, specifically engaging the alar cartilage and surrounding soft tissue of the nose. This external engagement point serves multiple functions: it provides tactile and visual confirmation of insertion depth, creates a mechanical lock that prevents rotational movement of the tubular body 102 within the nasal passage, and resists posterior migration of the device 100. The flat proximal end 104 remains positioned at the nostril opening, maintaining a flush interface with the nasal vestibule.

During normal operation, the internal lumen of the device 100 permits bidirectional airflow between the external environment and the nasopharynx. In circumstances where the distal opening becomes occluded by secretions, blood, or tissue apposition, the fenestrations 110 located proximal to the distal tip 106 provide alternative airflow pathways. This configuration maintains airway patency despite partial obstruction at the primary opening.

The standalone clip 208 is configured to interact with standard nasopharyngeal airways 200 already available in clinical settings. The clip aperture 212 is dimensioned to receive conventional nasopharyngeal airway devices 202, allowing the clip 208 to be applied to the external nose before or after nasopharyngeal airway insertion. Thus the separate clip 208 extends the securing and anti-rotation functionality to existing inventory without requiring replacement of current nasopharyngeal airway stock.

### Additional Considerations

As used herein any reference to “one embodiment” or “an embodiment” means that a particular element, feature, structure, or characteristic described in connection with the embodiment is included in at least one embodiment. The appearances of the phrase “in one embodiment” in various places in the specification are not necessarily all referring to the same embodiment.

Some embodiments may be described using the expression “coupled” and “connected” along with their derivatives. For example, some embodiments may be described using the term “coupled” to indicate that two or more elements are in direct physical or electrical contact. The term “coupled,” however, may also mean that two or more elements are not in direct contact with each other, but yet still co-operate or interact with each other. The embodiments are not limited in this context.

As used herein, the terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present) , and both A and B are true (or present) .

In addition, use of the “a” or “an” are employed to describe elements and components of the embodiments herein. This is done merely for convenience and to give a general sense of the invention. This description should be read to include one or at least one and the singular also includes the plural unless it is obvious that it is meant otherwise.

Upon reading this disclosure, those of skill in the art will appreciate still additional alternative structural and functional designs for a system and/or a process associated with the disclosed principles herein. Thus, while particular embodiments and applications have been illustrated and described, it is to be understood that the disclosed embodiments are not limited to the precise construction and components disclosed herein. Various apparent modifications, changes and variations may be made in the arrangement, operation and details of the method and apparatus disclosed herein without departing from the spirit and scope defined in the appended claims.

What is claimed is:

1. A nasopharyngeal airway device comprising:
a tubular body defining a hollow lumen extending from a proximal end to a distal end, the tubular body configured for insertion through a nostril of a patient to establish an airway passage through a nasopharynx of the patient;
a distal opening formed at the distal end of the tubular body, the distal opening having a beveled shape;
at least one fenestration formed in a wall of the tubular body at a location proximal to the distal opening, the at least one fenestration providing an auxiliary airflow passage through the wall of the tubular body; and
a securing clip coupled to the proximal end of the tubular body, the securing clip configured to engage external nasal anatomy of the patient to resist axial rotation of the tubular body and inhibit withdrawal of the tubular body from the nostril.

2. The nasopharyngeal airway device of claim 1, wherein the securing clip is integrally molded with the tubular body as a unitary structure.

3. The nasopharyngeal airway device of claim 1, wherein the securing clip is a separate component configured to be removably attached to the tubular body.

4. The nasopharyngeal airway device of claim 1, wherein the tubular body comprises an outer diameter that is uniform along an entire length of the tubular body.

5. The nasopharyngeal airway device of claim 1, wherein the securing clip has a position that creates a space between the securing clip and the tubular body, wherein the external nasal anatomy of the patient is positioned in the space when the distal end of the tubular body is positioned in the nasopharynx.

6. The nasopharyngeal airway device of claim 1, wherein the at least one fenestration comprises a plurality of fenestrations in the wall of the tubular body.

7. The nasopharyngeal airway device of claim 1, wherein the tubular body is semi-flexible and has a curved shape.

8. The nasopharyngeal airway device of claim 7, wherein the tubular body comprises a convex side and a concave side, and wherein the at least one fenestration is positioned on the convex side of the tubular body.

9. The nasopharyngeal airway device of claim 1, wherein the securing clip comprises a body portion that extends radially outward from the tubular body, and an engagement member that extends perpendicularly to the body portion, wherein the external nasal anatomy of the patient is frictionally engaged between the engagement member and the tubular body when the distal end of the tubular body is positioned in the nasopharynx.

10. A nasopharyngeal airway device comprising:
a tubular body having a proximal end with a flat terminal surface and a distal end with a beveled opening, the tubular body defining a hollow lumen therethrough for airflow;
a plurality of fenestrations formed through a wall of the tubular body at positions proximal to the beveled opening, the plurality of fenestrations configured to permit airflow when the beveled opening is obstructed; and
a securing clip coupled to the proximal end of the tubular body, the securing clip having a body portion that extends radially outward from the tubular body, and an engagement member that extends perpendicularly to the body portion, wherein external nasal tissue of a patient is frictionally engaged between the engagement member and the tubular body when the beveled opening is in a nasopharynx of the patient.

11. The nasopharyngeal airway device of claim 10, wherein the securing clip is integrally molded with the tubular body as a unitary structure.

12. The nasopharyngeal airway device of claim 10, wherein the securing clip is a separate component configured to be removably attached to the tubular body.

13. The nasopharyngeal airway device of claim 10, wherein the tubular body comprises an outer diameter that is uniform along an entire length of the tubular body.

14. The nasopharyngeal airway device of claim 10, wherein the tubular body is semi-flexible and has a curved shape.

15. The nasopharyngeal airway device of claim 14, wherein the tubular body comprises a convex side and a concave side, and wherein the plurality of fenestrations are positioned on the convex side of the tubular body.
